(19) United States
(12) Patent Application Publication
(10) Pub. No.: US 2007/0243623 A1
(43) Pub. Date: Oct. 18, 2007

(54) Device for the Presentation of Peptides or Proteins, Method for the Preparation and Use Thereof

(75) Inventors: Oleg Melnyk, Annoeullin (FR); Xavier Duburcq, Lille (FR); Helene Gras-Masse, Merignies (FR); Christophe Olivier, Lille (FR); Fengling Zhou, Lille (FR); Claude Auriault, Nomain (FR); Ahmed Bouzidi, Annoeullin (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: Sedac Therapeutics, Loos (FR); Centre National De La Recherche Scientifique, Paris (FR); Universite De Lille 2, Lille (FR)

(21) Appl. No.: 10/515,190

(22) PCT Filed: May 15, 2003
(86) PCT No.: PCT/FR03/01471

§ 371 Date: Feb. 13, 2007

(30) Foreign Application Priority Data
May 28, 2002 (FR) 02/06489

(51) Int. Cl. G01N 33/48; B01L 11/00; G01N 1/28; G01N 1/36

(52) U.S. Cl. 436/86; 422/243; 436/174; 73/864.91

(57) ABSTRACT

The invention relates to a device for the presentation of polypeptides, a method for the preparation and the use thereof, as diagnostic tool (polypeptide chip) for miniaturized and highly parallel detection of structurally or functionally complementary molecules of said polypeptides, i.e. antibodies.

The present invention relates to a device for the presentation of polypeptides, to its method of preparation and its uses, in particular as a diagnostic tool (polypeptide chip) for the miniaturized detection of molecules which are structurally or functionally complementary to said polypeptides, in particular antibodies.

For the purposes of the present invention, the term “polypeptide chip” corresponds to the English terms “peptide arrays”, “peptide microarrays”, “peptide chips”, “protein chips” or “protein arrays”, commonly used in the literature.

There is currently great enthusiasm for the use of polypeptide chips allowing the detection, in various liquid biological media, of antibodies or of specific parts thereof, of antigens (in particular viral, bacterial or parasitic antigens), of receptors, of sequences responsible for binding to a molecule (enzyme, receptor, antibody), the study of the specificity of enzymes or the development of artificial receptors.

Now, in this specific context, it is essential to be able to have available polypeptide chips having a number of qualities.

These chips must in particular allow the reproducible immobilization of probes, since a reproducible immobilization is a condition for a detection which is itself reproducible. For the purposes of the present invention, the expression probe is understood to mean any polypeptide which is deposited at the surface of a support and which serves for the capture of targets present in a biological medium, these probes being specific for the targets to be detected.

These chips must also allow the sensitive detection of the targets or complementary receptors contained in the biological medium. The sensitivity of detection depends on the level of immobilization, the level of capture and the method of detecting a signal, but also and especially on the level of background noise (nonspecific signal). A reduction in the background noise improves the signal/noise ratio. Indeed, in a device in which the presence of biological species in the vicinity of the surface is detected, the background noise comes essentially from the nonspecific adsorption of molecules other than the biological species of interest which it is desired to detect, and which should consequently be limited. It would therefore be ideal to obtain a device which possesses a very low background noise and a high signal detection intensity.

Moreover, from an industrial point of view, it is advantageous to be able to have devices which can be prepared in a simple manner and which have an excellent stability during storage before use.

Currently, there are mainly two main methods of preparing polypeptide chips using polypeptides prepared ex situ.

The first main method consists in binding the polypeptides in a covalent manner to a solid surface such as a glass or an organic polymer surface. Accordingly, there has already been proposed for example in the article by G. MacBeath et al., Science, 2000, 289, 1760-1763, a method of preparation consisting in immobilizing the polypeptides via an imine bond resulting from the reaction between an amine functional group of the polypeptides and an aldehyde functional group of a silanized support. This method then requires a step for reducing in situ the imine functional group, for example with sodium borohydride in order to stabilize the polypeptide-surface linkage. The major disadvantage of this approach is the need to carry out chemical steps for linking the polypeptide to the surface of the support, which may cause in particular, in some cases, degradation of the polypeptide used. Furthermore, such reactions significantly complicate the method for manufacturing polypeptide chips.

The second main method consists in immobilizing the polypeptides by adsorption onto a surface without establishing a covalent linkage. This method quite obviously has the advantage of being simpler from an industrial point of view since it does not necessarily involve a chemical step of functionalizing the polypeptides. Accordingly, the article by Falipou S. et al., Bioconjugate Chem., 1999, 10, 346-353 describes a method according to which SiO₂ beads or glass slides are silanized with 3-cyanopropyldimethylchlorosilane so as to allow immobilization of antibodies (glycosylated proteins), the noncovalent attachment taking place, in this case, via hydroxyl functional groups of the glycosylated parts. This method nevertheless has the disadvantage of being limited to glycosylated proteins. It has moreover already been proposed, in particular in international application WO 00/63701, to immobilize polypeptides onto glass surfaces coated with a cationic polymer such as polylysine, by means of electrostatic bonds. However, even before the immobilization of the polypeptides, the properties of the glass slides thus prepared change over time. Also, the devices manufactured from this type of support are unstable over time (variation of the signal according to the degree of aging of the device). Finally, these surfaces lead to a high background noise level (Haab B B. et al., Genome Biology, 2001, research 0004.1-13).

The inventors therefore set themselves the aim of overcoming all the problems encountered with the prior art devices described above and to provide devices for the presentation of polypeptides which are stable over time (at least 3 months of aging under accelerated conditions, which corresponds to 12 months of aging at room temperature), simple to manufacture and to use, applicable to any type of polypeptides without the need for steps to functionalize them and which allow sensitive and reproducible detection of a signal with a very low background noise.

The first subject of the present invention is therefore a device for the presentation of polypeptides, characterized in that it comprises at least one solid support functionalized with semicarbazide groups onto which said polypeptides are adsorbed.

Whereas surfaces comprising semicarbazide groups are normally used to immobilize biomolecules such as nucleic acids functionalized with benzaldehyde groups (Podyminogin M A et al., Nucleic Acid Research, 2001, 29, 5090-5098) or polypeptides modified with α-oxoaldehyde functional groups (international application WO 01/42495) by forming a semicarbazone-type covalent bond, the inventors have observed, surprisingly, that the use of these surfaces also makes it possible to immobilize polypeptides by mere adsorption, in a lasting and stable manner over time, without the need to functionalize them beforehand.

Thus, the mere adsorption of polypeptides onto such a support makes it possible to obtain devices which are stable over time (at least 12 months at room temperature), simple to manufacture and to use, according to a methodology which is applicable to any type of polypeptides (without adding chemical reagents, apart from the buffers conventionally used for solubilizing polypeptides) and which allow sensitive and reproducible detection of a signal with a very low background noise.

For the purposes of the present invention, the general term of “polypeptides” denotes all the peptides comprising at least two amino acids (of the L or D series, alpha-amino acids, beta-amino acids, alpha-hydrazino acids, alpha-amino acids which may or may not be proteinogenic), peptidomimetics (mimics of secondary structures, mimics of beta-elbow for example), proteins and protein fragments.

The polypeptides adsorbed at the surface of the device in accordance with the present invention may be polypeptides from extraction or recombinant polypeptides, without constraint as to their structure (polypeptides with or without post-translational modification). They may also be synthetic polypeptides carrying various modifications, such as for example a polyethylene glycol group or chemical groups which are inert toward the surface semicarbazide groups, such as for example semicarbazone and hydrazone groups.

The deposition of the polypeptides on the semicarbazide support is accompanied by their spontaneous adsorption onto the support.

Any solid supports which may be functionalized with a semicarbazide group can be used according to the invention. Among such supports, there may be mentioned in particular organic or inorganic materials chosen for example from glass, silicon and its derivatives and synthetic polymers.

The supports functionalized with semicarbazide groups may be imprinted for example with a pin “spotter”.

Thus, the immobilization strategy:

is simple at the experimental level and is highly reproducible;

applies to any type of polypeptide which is nonfunctionalized or functionalized with a group which is chemically inert toward the semicarbazide groups of the support;

uses surfaces which are functionalized with a stable, nonhydrolyzable functional group;

makes it possible to obtain a high density of adsorption of the polypeptides at the surface of the support, ensuring a very high signal/background noise ratio (typically the background noise represents 0.1% of the signal detected).

The quality of the adsorption of the polypeptides onto the support (density, homogeneity) may be controlled by its capacity to bind a fluorescent synthetic peptide probe.

The subject of the invention is also a method for preparing the devices for the presentation of polypeptides as described above, comprising the following steps:

the functionalization of a solid support with semicarbazide groups, and

the deposition, in the form of spots, of samples of polypeptides and their adsorption onto the support thus functionalized.

According to a first variant of this method, the functionalization of the support comprises:

the introduction of an amine functional group by a reaction for silanization of the support,

the conversion of the amine functional group to an isocyanate functional group,

the reaction of the isocyanate functional group with a hydrazine derivative in order to form the semicarbazide group.

According to a second variant of this method, the functionalization of the support is performed in a single step by reacting the support with a silane carrying a semicarbazide group.

The step of depositing the polypeptides preferably comprises:

the preparation of polypeptide solutions in a buffer, at a concentration of between 10 mg/ml and 0.01 mg/ml;

their distribution into a container appropriate for their collection, of the microtiter plate well type;

their collection with the aid of a manual or automated sample collecting apparatus, for example of the “spotter” type;

their deposition onto the semicarbazide support; and optionally

the saturation of the support.

At the end of their preparation, the devices in accordance with the invention may be used directly or stored at room temperature, protected from light and from dust.

The subject of the invention is also the use of the devices for the presentation of polypeptides as “polypeptide chips”, as a miniaturized and highly parallel diagnostic tool, or for the detection of a risk during transfusion or organ donation.

The devices in accordance with the present invention may also be used as “polypeptide chips” for the serotyping or screening of epitopes.

These uses involve the detection of antigen-antibody type responses by the use of labeled, fluorescent, radioactive or chemically labeled reagents.

The devices in accordance with the present invention may also be used as polypeptide chips for the quantification of proteins in complex biological media.

Finally, the devices in accordance with the present invention may also be used for analyzing the relationships between peptide biological molecules of the ligand-receptor type.

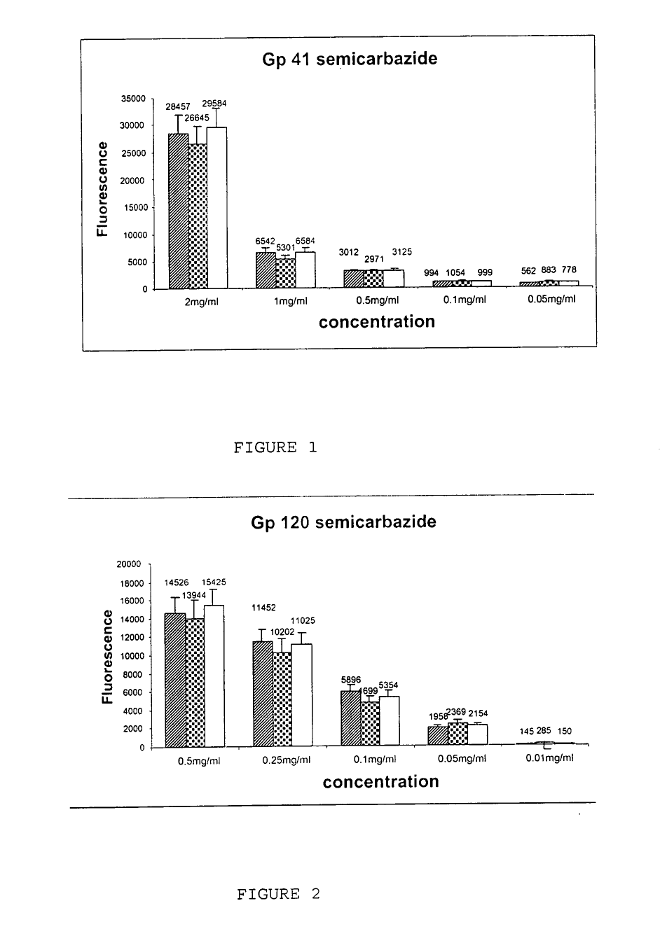

In addition to the preceding features, the invention also comprises other features which will emerge from the description which follows, which refers to an example of preparation of glass slides functionalized with semicarbazide groups, to an example detailing the protocol for adsorption of polypeptides onto glass slides semicarbazides and the use of the corresponding devices thus obtained, for use of the devices of the invention for the serodiagnosis of hepatitis B, hepatitis C and AIDS, for studying the stability of these devices, for studying multiserodetection and for the detection of the hepatitis B surface antigen, and to the appended FIGS. 1 to 5 in which:

FIGS. 1 and 2 represent the fluorescence signal measured as a function of the antigen concentration, after 0, 1 or 3 months of aging of semicarbazide slides onto which AIDS virus antigens have been adsorbed;

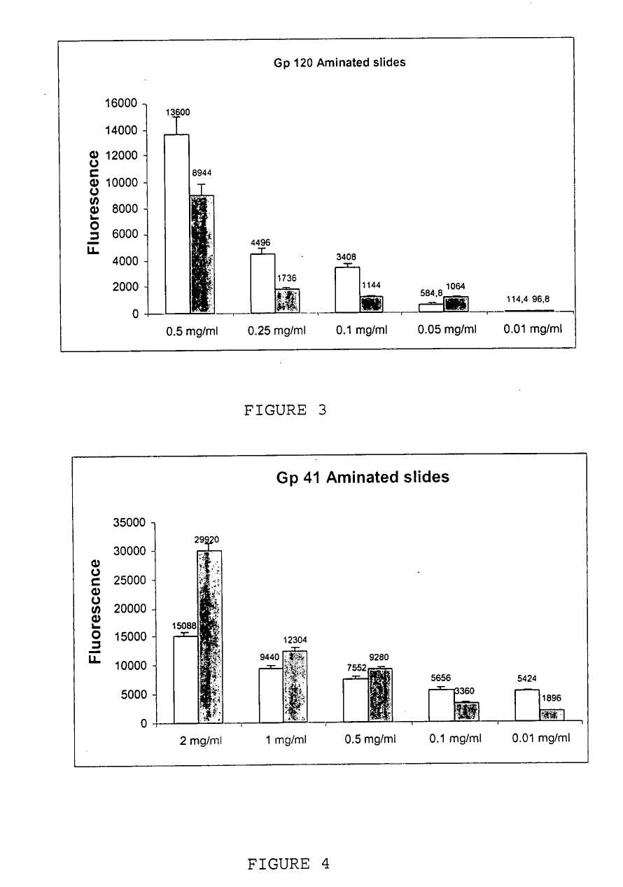

FIGS. 3 and 4 represent the fluorescence signal measured for aminated slides to which AIDS virus antigens have been attached, as a function of the concentration and after 0 and 1 month of accelerated aging;

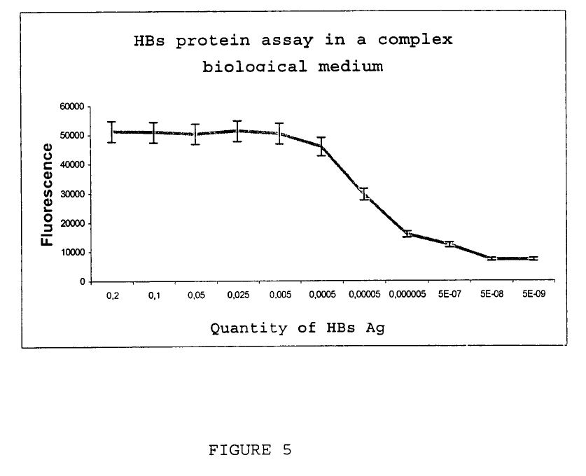

FIG. 5 represents the fluorescence measured as a function of the quantity of a recombinant HBs antigen in a test for serodetection of the hepatitis B surface antigen.

### EXAMPLE 1

### Functionalization of Glass Slides with Semicarbazide Groups

This functionalization was carried out according to two variants.

1) First Variant

a) Step A: Washing, Stripping and Silanization

Precleaned commercial microscope slides (Esco), with round edges and with a depolished margin are immersed in a solution consisting of a mixture of hydrogen peroxide and sulfuric acid (50/50, v/v) overnight. Preliminary three-minute rinses are performed with deionized water (3 times) and then with methanol (once), before immersing the slides in a bath containing 3% aminopropyltrimethoxysilane in methanol at 95% for 30 minutes under ultrasound. The slides are then rinsed successively with baths of 3 minutes in methanol (once), with deionized water (twice) and finally with methanol (once). The slides are then drained for a few minutes, dried for 15 minutes in an oven at 110° C., and then stored in a desiccator under vacuum.

b) Step B: Formation of an Isocyanate

The previously silanized slides are immersed for 2 hours in a solution of 1,2-dichloroethane containing triphosgene (100 mmol/l) and diisopropyl-ethylamine (DIEA) (800 mmol/l).

c) Step C: Functionalization with Semicarbazide Groups

The slides obtained in step b) above are then rapidly drained before being directly immersed in a solution containing 9-fluorenylmethoxycarbonyl-NH—NH₂ (Fmoc-NH—NH₂), prepared beforehand according to Zhang et al., Anal. Biochem., 1991, 195, 160-170, at 22 mmol/l in dimethylformamide (DMF) and treated for 2 hours with ultrasound. The slides are then rinsed successively with two baths of 3 minutes in DMF.

d) Step D: Deprotection

The slides previously obtained in step c) above are immersed in a solution of DMF containing piperidine (0.2% by volume) and 1,8-diazabicyclo[5.4.0]-undec-7-ene (DBU) (2% by volume) for 30 minutes. The slides are then rinsed successively with baths of 3 minutes in DMF (once), with deionized water (twice) and finally with methanol (once) before being dried and stored in a desiccator under vacuum.

2) Second Variant

The steps of silanization and functionalization with semicarbazide groups are coupled in a single reaction, by prior preparation of the reagent.

a) Step A: Preparation of the Silanization Reagent Containing the Protected Semicarbazide Group (Fmoc-NH—NH—CO—NH— (CH₂)₃—Si—(OEt)₃)

515 mg of Fmoc-NH—NH₂ (2.03 mmol) are suspended in 15 ml of absolute ethanol. The mixture is heated to reflux temperature (75-80° C.). 570 ml of isocyanopropyltriethoxysilane (2.28 mmol, 1.2 eq) are then added all at once. After the disappearance of the suspension (15-20 minutes), the ethanol is evaporated off. The white solid obtained is dissolved in a minimum of dry dichloromethane, and then precipitated using dry pentane. After filtration under argon, 841 mg (83%) of a pure solid are recovered.

b) Step B: Preparation of the Slides

Precleaned commercial microscope slides (Esco), with round edges and with a depolished margin are immersed in a solution consisting of a mixture of hydrogen peroxide and sulfuric acid (50/50, v/v) overnight. The slides are then rinsed, with stirring, in the following successive baths: deionized water (3 times 3 minutes), absolute ethanol (once 3 minutes) and then dried using a slide vane rotary vacuum pump.

The slides are then immersed for 2 hours in a solution containing 1 mg/ml of the silanization reagent prepared above in step a) in a mixture of tetrahydrofuran (THF) at 10% in toluene at 47° C. and under ultrasound. The slides are then rinsed, with stirring, in toluene (twice 3 minutes) before being drained, and then dried for 15 minutes in an oven at 120° C. and stored in a desiccator under vacuum.

c) Step C: Deprotection

The slides previously obtained above in step b) are immersed in a solution of DMF for 3 minutes before being placed under stirring in a bath containing piperidine (0.2% by volume) and diazabicycloundecene (2% by volume) in DMF for 30 minutes, with stirring. The slides are then rinsed successively with baths of 3 minutes in DMF (once), in deionized water (twice) and finally in methanol (once) before being dried and stored in a desiccator under vacuum.

The slides thus prepared are ready to be used to adsorb polypeptides.

### EXAMPLE 2

### Protocol for Using the Semicarbazide Slides

1) Adsorption of the Polypeptides onto the Slides

Solutions of synthetic peptides or of recombinant proteins (antigens) are diluted, between 0.1 and 10 mg/ml according to the protein used, in a phosphate buffer (pH 7.4) or in a carbonate/bicarbonate buffer (pH 9.2) and are then distributed into the wells of a 96-well ELISA plate. A minimum volume of 20 μl of sample per well is necessary to carry out the step of imprinting the slides.

The plate is then introduced into a 4-pin automated spotter (for example of the MWG 417 Arrayer type from the company Affymetrix). The apparatus which makes it possible to carry out the depositions of antigen solutions is called spotter.

The slides are then imprinted with the solutions of polypeptides according to a preestablished scheme. The pins are then thoroughly washed according to the recommendations of the manufacturer.

2) Storage of the Slides

The slides thus imprinted are stored at room temperature, in a closed cupboard, protected from light and from dust. The stability of the slides was studied (see example 6 below). The results show that the properties of the slides thus imprinted are not impaired during a three-month storage at 37° C. in a humid atmosphere (accelerated aging conditions).

3) Visualization of the Slides

The imprinted slides are first of all subjected to a sonication step for 1 hour in a saturation solution: phosphate buffer supplemented with 1.8% NaCl, pH 7.4 (PBS solution)+0.1% Tween® 20+5% skimmed milk).

The slides are then subjected to three successive washes with a PBS solution in the presence of 0.05% Tween® 20.

The slides are then incubated for 45 minutes at 37° C. in the presence of 100 μl of serum from patients, diluted 1/50 in a dilution buffer (PBS solution+0.1% Tween® 20+5% skimmed milk) under a glass coverslip (24×60 mm). The incubation is performed in a humid atmosphere.

Following this incubation, three successive washes of the slides with a PBS solution supplemented with 0.05% Tween® 20 are then performed.

The step for detection of the patients' antibodies on the polypeptides adsorbed onto the slides is carried out by attaching thereto fluorescent anti-human Ig antibodies: 100 μl of anti-human IgG-A-M solution of antibodies labeled with rhodamine (TRITC) (Jackson ImmunoResearch Laboratories, Baltimore, USA), diluted 1/100 in dilution buffer (PBS solution+0.1% Tween® 20+5% skimmed milk). Each slide is then covered with a glass coverslip (24×60 mm) and left to incubate for 45 minutes at 37° C. under a humid atmosphere.

Following this incubation, a series of 3 successive washes of the slides with a solution of PBS supplemented with 0.05% Tween® 20 is then performed. These washes are followed by rinsing of the slides with distilled water and by drying the slides with ethanol.

The reading of the slides is then performed by measuring the fluorescence emitted with the aid of a slide scanner (Affymetrix 418 Array Scanner), at two different power settings (P35/PMT 50 or P55/PMT 70). The quantification of the fluorescence emitted is then performed with the aid of the Scanalyse® software (Stanford-University Software). Unless otherwise stated, the results presented in the tables below were obtained at the power P35/PMT 50.

All the examples which follow were carried out according to this protocol.

### EXAMPLE 3

### Use of Polypeptide Chips According to the Invention for the Serodiagnosis of Hepatitis B

1) Materials and Methods

All the depositions were performed in duplicate on slides as detailed above in example 1, using an MWG 417 Arrayer Spotter (Affymetrix). The distance between the deposits is 375 μm, the control for the efficacy of the washing of the pins is performed by deposition of water, which shows by fluorescence no trace of antigen.

In this example, the glass slides were imprinted with various hepatitis B virus antigens. The terms surface (HBs), core (HBc) or (HBe) correspond to various regions of this virus. Twenty depositions per antigen and per concentration were performed.

HBs antigen, two batches were used:

HBs batch a: antigen marketed by the company Advanced Immuno Chemical under the reference A1-HS7, at 3 mg/ml.

HBs batch b*: 5 mg/ml.

Hbe antigen*: 10 mg/ml.

HBc antigen*: 10 mg/ml.

Positive control*: protein A

The HBV antigens (HBs batch b, Hbe, HBc) and HCV antigens were produced by “The Hepatopathy Research Institute”, Pediatric Hospital of Beijing (Beijing, China).

Sera from patients whose serology is known (that is to say verified with a reference ELISA test) were analyzed using the chips in accordance with the invention.

The preparation of the sera were as follows:

serodiagnostic HBs Ag with batch a: 40 sera noted positive and 40 sera noted negative;

serodiagnostic HBs Ag with batch b: 40 sera noted positive and 40 sera noted negative;

serodiagnostic Hbe: 16 sera noted positive and 16 sera noted negative;

serodiagnostic Hbc: 60 sera noted positive and 40 sera noted negative.

2) Results

the results obtained for the positive sera are present in table 1 below. In this table, the values given correspond to the fluorescence value less the mean background noise for the slide.

TABLE I

| | | | | Test on biochips (quantity | | | |
|---|---|---|---|---|---|---|---|
| | | | | of fluorescence) | | | |
| | Reference ELISA test | | | HBs | HBs | | |
| Serum | HBs | HBe | HBc | Batch a | Batch b | HBe | HBc |
| 1 | + | + | + | 5482 | 5548 | 10254 | 15049 |
| 2 | + | nt * | + | 4521 | 7845 | nt | 16804 |
| 3 | + | nt | + | 2899 | 5784 | nt | 25014 |
| 4 | + | + | + | 4503 | 9854 | 5784 | 11568 |
| 5 | + | + | + | 4310 | 8547 | 14215 | 19580 |
| 6 | + | nt | + | 6854 | 7548 | nt | 28954 |
| 7 | + | + | + | 5871 | 10055 | 8457 | 9109 |
| 8 | + | nt | + | 3771 | 7156 | nt | 14201 |
| 9 | + | + | + | 4587 | 8457 | 11451 | 21131 |
| 10 | + | nt | + | 5684 | 11784 | nt | 9605 |
| 11 | + | + | + | 4089 | 9524 | 10254 | 15460 |
| 12 | + | nt | + | 3854 | 9995 | nt | 12541 |
| 13 | + | + | + | 2998 | 8457 | 11254 | 47988 |
| 14 | + | nt | + | 7033 | 9854 | nt | 25153 |
| 15 | + | + | + | 5714 | 12541 | 11116 | 27405 |
| 16 | + | nt | + | 3601 | 6985 | nt | 18954 |
| 17 | + | nt | + | 4707 | 7722 | nt | 14521 |
| 18 | + | + | + | 5515 | 8146 | 7895 | 25254 |
| 19 | + | nt | + | 6939 | 9524 | nt | 10939 |
| 20 | + | nt | + | 4196 | 10091 | nt | 34137 |
| 21 | + | nt | + | 5553 | 9104 | nt | 36387 |
| 22 | + | nt | + | 4357 | 9182 | nt | 22703 |
| 23 | + | + | + | 4007 | 10541 | 9548 | 31225 |
| 24 | + | nt | + | 4739 | 16608 | nt | 36695 |
| 25 | + | nt | + | 3958 | 12541 | nt | 23552 |
| 26 | + | nt | + | 5270 | 8620 | nt | 11251 |
| 27 | + | nt | + | 3254 | 9288 | nt | 57835 |
| 28 | + | + | + | 4194 | 11415 | 7854 | 18828 |
| 29 | + | nt | + | 3997 | 7698 | nt | 28461 |
| 30 | + | nt | + | 5980 | 8145 | nt | 35261 |
| 31 | + | nt | + | 3215 | 9354 | nt | 15854 |
| 32 | + | nt | + | 4254 | 7125 | nt | 28554 |
| 33 | + | + | + | 5412 | 16859 | 5896 | 26551 |
| 34 | + | nt | + | 3984 | 8327 | nt | 29376 |
| 35 | + | nt | + | 4521 | 10270 | nt | 15189 |
| 36 | + | nt | + | 4879 | 7458 | nt | 29276 |
| 37 | + | nt | + | 5895 | 17791 | nt | 25654 |
| 38 | + | nt | + | 4987 | 13232 | nt | 29854 |
| 39 | + | nt | + | 6587 | 14587 | nt | 21900 |
| 40 | + | nt | + | 3254 | 9587 | nt | 15854 |
| 41 | + | nt | + | nt | 9854 | nt | 25044 |
| 42 | + | nt | + | nt | 9968 | nt | 19578 |
| 43 | + | + | + | nt | 13263 | 4985 | 25654 |
| 44 | + | nt | + | nt | 12335 | nt | 21066 |
| 45 | + | + | + | nt | 11009 | 14882 | 25654 |
| 46 | + | nt | + | nt | 14528 | nt | 19850 |
| 47 | + | nt | + | nt | 7458 | nt | 9854 |
| 48 | + | nt | + | nt | 8954 | nt | 14592 |
| 49 | + | nt | + | nt | 9958 | nt | 15253 |
| 50 | + | + | + | nt | 10499 | 7180 | 12454 |
| 51 | + | nt | + | nt | 8521 | nt | 14254 |
| 52 | + | nt | + | nt | 8637 | nt | 21548 |
| 53 | + | nt | + | nt | 7410 | nt | 35689 |
| 54 | + | nt | + | nt | 9587 | nt | 14587 |
| 55 | + | nt | + | nt | 5985 | nt | 16548 |
| 56 | + | + | + | nt | 8457 | 4958 | 25415 |
| 57 | + | nt | + | nt | 9485 | nt | 39548 |
| 58 | + | nt | + | nt | 8567 | nt | 9854 |
| 59 | + | nt | + | nt | 15421 | nt | 12458 |
| 60 | + | nt | + | nt | 8214 | nt | 19874 |
| Threshold | | | | 421 | 784 | 712 | 854 |
| value | | | | | | | |

* nt means not tested

The threshold value (VS) is calculated as the mean of the signal for 20 negative sera minus the background noise to which is added 3 times the standard deviation calculated on these negative serum values. All the sera noted negative gave a fluorescence value less than the threshold value and were therefore indeed found to be negative using the slides in accordance with the invention.

All these results show that the use of polypeptide chips in accordance with the invention allows a serodiagnosis which is 100% sensitive and specific for all the hepatitis B antigens tested on the various sera. Indeed, all the sera recorded as being positive were detected, whereas none of the sera recorded as being negative were found to be falsely positive by this method.

### EXAMPLE 4

### Use of Polypeptide Chips According to the Invention for the Serodiagnosis of Heptatitis C

1) Materials and Methods

According to the method described above in example 3, glass slides were imprinted with various hepatitis C virus antigens, at various concentrations. The terms NS3, NS4 or core correspond to the various regions of the hepatitis C virus. In this example, protein A was used as positive control.

HCV Batch 1 corresponding to the whole antigen: depositions at 0.5 mg/ml, 0.1 mg/ml and 0.05 mg/ml;

HCV Batch 2 corresponding to the whole antigen: depositions at 2 mg/ml, 1 mg/ml and 0.5 mg/ml;

HCV core Ag: depositions at 1 mg/ml, 0.5 mg/ml and 0.1 mg/ml;

HCV NS3 Ag Batch 3: deposition at 1 mg/ml, 0.5 mg/ml and 0.1 mg/ml;

HCV NS4 Ag Batch 5: depositions at 0.4 mg/ml and 0.1 mg/ml;

100 sera noted positive by a conventional 3.0 Abbot EIA test (ELISA type method) and 30 sera noted negative by the same method were tested in this example.

2) Results

The results made it possible to determine the optimum concentrations to use for each antigen:

HCV Batch 2: 2 mg/ml

HCV NS3 Ag Batch 3: 0.5 mg/ml

HCV NS4 Ag Batch 5: 0.4 mg/ml

HCV core Ag: 1 mg/ml

The results obtained are presented in table II. In this table, the values given correspond to the fluorescence value minus the mean background noise for the slide.

TABLE II

| | RIBA | | | | | HCV Batch 1 | HCV Batch 2 | HCV core Ag | NS4 HCV Ag | NS3 HCV Ag |
|---|---|---|---|---|---|---|---|---|---|---|
| Serum | NS5 | Core | NS3 | NS4 | Abbott | 0.5 mg/ml | 2 mg/ml | 1 mg/ml | 0.4 mg/ml | 0.5 mg/ml |
| 1 | | +++ | ++ | ++ | 117 | 11254 | 35268 | 17854 | 6254 | 13215 |
| 2 | − | − | − | − | 1 | 25 | 156 | 0 | 0 | 0 |
| 3 | | − | +++ | +++ | 125 | 11245 | 39421 | 0 | 10545 | 21514 |
| 4 | | − | ++ | + | 50 | 10524 | 32157 | 0 | 5214 | 8954 |
| 5 | | − | +++ | +++ | ? | 14521 | 39564 | 0 | 11245 | 15462 |
| 6 | 0.5+ | − | − | 0.5+ | 2 | 331 | 1085 | 0 | 312 | 0 |
| 7 | | ++ | + | + | 30 | 8547 | 15242 | 15421 | 5985 | 9584 |
| 8 | | − | ++ | − | 1 | 456 | 1025 | 0 | 0 | 452 |
| 9 | | − | − | − | 2 | 42 | 135 | 0 | 0 | 0 |
| 10 | | +++ | ++ | ++ | 112 | 14215 | 38546 | 21542 | 12352 | 20135 |
| 11 | | +++ | ++ | ++ | 46 | 17548 | 41526 | 23215 | 17548 | 21524 |
| 12 | + | 0.5+ | − | − | 7 | 925 | 3854 | 4162 | 0 | 0 |
| 13 | | ++ | + | ++ | 105 | 14582 | 35216 | 18546 | 13250 | 15468 |
| 14 | 0.5+ | − | 0.5+ | − | 1 | 495 | 1958 | 0 | 0 | 568 |
| 15 | | +++ | ++ | 0.5+ | ? | 10254 | 29856 | 21452 | 2154 | 7548 |
| 16 | | ++ | +++ | +++ | 130 | 15429 | 32964 | 29584 | 12496 | 16548 |
| 17 | | − | − | − | 1 | 29 | 107 | 0 | 0 | 0 |
| 18 | | − | − | − | 2 | 36 | 185 | 0 | 0 | 0 |
| 19 | | +++ | +++ | +++ | 113 | 17859 | 39520 | 28964 | 15421 | 19548 |
| 20 | | +++ | ++ | +++ | 100 | 21524 | 451214 | 31250 | 19854 | 20482 |
| 21 | | +++ | +++ | + | 74 | 19854 | 35219 | 28546 | 5485 | 17458 |
| 22 | | 0.5+ | + | 0.5+ | ? | 512 | 1021 | 456 | 651 | 325 |
| 23 | | +++ | ++ | 0.5+ | 80 | 15214 | 38546 | 21549 | 542 | 12415 |
| 24 | | − | ++ | + | 25 | 5684 | 12409 | 0 | 4632 | 6895 |
| 25 | | − | − | − | 1 | 47 | 146 | 0 | 0 | 0 |
| 26 | | − | − | − | 73 | 28 | 134 | 0 | 0 | 0 |
| 27 | | +++ | +++ | ++ | 78 | 18549 | 39507 | 25348 | 8549 | 17965 |
| 28 | | +++ | ++ | ++ | ? | 21549 | 41305 | 29846 | 14528 | 22304 |
| 29 | | − | − | − | 1 | 51 | 152 | 0 | 0 | 0 |
| 30 | | ++ | ++ | − | ? | 12045 | 25365 | 18546 | 0 | 4325 |
| 31 | 0.5+ | − | + | − | 4 | 1524 | 6528 | 0 | 0 | 1095 |
| 32 | | − | − | − | 6 | 39 | 128 | 0 | 0 | 0 |
| 33 | | +++ | +++ | 0.5+ | 142 | 9602 | 21795 | 11542 | 598 | 5695 |
| 34 | | +++ | +++ | +++ | 120 | 16325 | 35985 | 24065 | 11248 | 16524 |
| 35 | | +++ | +++ | +++ | 159 | 25639 | 45681 | 29032 | 15486 | 21468 |
| 36 | | +++ | +++ | ++ | 135 | 21025 | 32598 | 25584 | 12495 | 15421 |
| 37 | | +++ | + | − | ? | 15421 | 25625 | 20135 | 0 | 2154 |
| 38 | | +++ | +++ | +++ | 140 | 24569 | 38569 | 29584 | 18542 | 14025 |
| 39 | | +++ | +++ | 0.5+ | 132 | 12542 | 25146 | 14201 | 354 | 5698 |
| 40 | | +++ | +++ | +++ | ? | 19854 | 29524 | 21024 | 11245 | 9587 |

| | | | | | |
|---|---|---|---|---|---|
| Threshold value: | 115 | 315 | 0 | 0 | 0 |

NB: In this table, the question mark means that the Abbott test was not performed.

All the sera noted positive were found to be positive using the slides in accordance with the invention.

In the same manner, all the sera noted negative were found to be negative using the slides in accordance with the invention.

The results show that the fluorescence signal obtained at a low power setting (P35, PMT50) is high and does not require a second reading at a higher scanner power setting (L55, PMT 70).

For HCV Batch 1 and Batch 2, for which a low fluorescence value is observed for the negatives, the threshold value is calculated as the mean of 20 sera minus the background noise, supplemented with 3 times the standard deviation on these values for the negative sera. For the other antigens tested, no fluorescence greater than the background noise is observed for the negative sera; the threshold value therefore corresponds to the value for the negatives minus the background noise, that is zero.

Thus, for HCV core, NS4 and NS3, if a fluorescence signal is observed, it means that the serum is positive. This represents a huge advantage compared with a serodiagnosis performed in a conventional manner by the ELISA method for which a nonspecific signal is always observed with the negative sera, which requires defining a threshold value which depends on this nonspecific signal.

Furthermore, it is evident from these results that Batch 2 is of particular interest: it increases the sensitivity of the serodetection by making it possible to unambiguously detect sera which are weak by Abbott ELISA (less than 10), for which RIBA (Recombinant Immunoblot Assay: recombinant immunoblot test performed on a nitrocellulose membrane and which makes it possible to know against which hepatitis C virus antigen the antibodies produced following a viral infection are directed) is either positive or borderline (0.5+). The use of the antigen of Batch 2 adsorbed onto a semicarbazide support therefore makes it possible to improve the detection sensitivity compared with ELISA, which is particularly advantageous in terms of how early the detection is made. Furthermore, the polypeptide biochips in accordance with the invention are more specific than the reference Abbott ELISA. Indeed, the Abbott false-positive sera (sera 2, 9, 17, 18, 25, 26, 29 and 32, sera confirmed RIBA negative) were correctly diagnosed (negative) using the polypeptide biochips in accordance with the invention.

The semicarbazide glass slides onto which NS3, NS4 and core antigens are adsorbed therefore constitute sensitive and specific diagnostic tools which make it possible to differentiate the different hepatitis C antigens whereas with the ELISA technique, which uses combinations of different antigens, it is not possible to obtain such a differentiation.

### EXAMPLE 5

### Use of Polypeptide Chips According to the Invention for the Serodiagnosis of AIDS

1) Materials and Methods

In this example, 30 depositions per antigen were performed on each slide.

Two different antigens of the AIDS virus (HIV) were tested; they are the Gp41 and Gp120 antigens which are envelope glycoproteins of the virus. These HIV antigens are produced by Lily Bioproducts, Hanan, China.

Protein A was used as positive control.

The concentrations tested are the following:

Gp41: 2; 1; 0.5; 0.1 and 0.05 mg/ml.

Gp120: 0.5; 0.25; 0.1 and 0.05 mg/ml.

90 sera noted positive and 20 sera noted negative were tested (ELISA technique: Genscreen Plus® HIV Ab tests from the company BIORAD and HIV Integral test from the company BEHRING).

2) Results

The results obtained are presented in table III below. In this table, the values given correspond to the fluorescence value minus the mean background noise for the slide.

TABLE III

| Biochips | |
|---|---|
| Gp 120 | Gp 41 |

| Serum | 0.5 mg/ml | 0.25 mg/ml | 0.1 mg/ml | 0.05 mg/ml | 2 mg/ml | 1 mg/ml | 0.5 mg/ml | 0.1 mg/ml | 0.05 mg/ml | ELISA |
|---|---|---|---|---|---|---|---|---|---|---|
| 1 | 41730 | 45803 | 10773 | 7431 | 31803 | 27549 | 22958 | 13036 | 8696 | positive |
| 2 | 41251 | 6674 | 8697 | 9715 | 8801 | 17586 | 9713 | 4403 | 2753 | positive |
| 3 | 30864 | 25786 | 11666 | 10212 | 17026 | 29694 | 24347 | 23607 | 18137 | positive |
| 4 | 32520 | 32206 | 11738 | 15973 | 11655 | 17779 | 12531 | 6333 | 1276 | positive |
| 5 | 42022 | 28683 | 11415 | 12791 | 16405 | 15092 | 8320 | 2425 | 416 | positive |
| 6 | 38148 | 28426 | 18753 | 12226 | 19196 | 21076 | 16184 | 13239 | 10817 | positive |
| 7 | 40494 | 30883 | 10035 | 9086 | 6827 | 23866 | 10504 | 9898 | 9131 | positive |
| 8 | 20017 | 10248 | 4870 | 1697 | 2542 | 4125 | 1952 | 1476 | 624 | positive |
| 9 | 39166 | 33241 | 18446 | 19500 | 32768 | 31779 | 16132 | 31833 | 22971 | positive |
| 10 | 30095 | 31628 | 28099 | 9779 | 26105 | 9199 | 10716 | 24191 | 17998 | positive |
| 11 | 25325 | 32705 | 20765 | 6586 | 24592 | 13347 | 13756 | 17951 | 17910 | positive |
| 12 | 31086 | 29523 | 20547 | 23421 | 31727 | 9113 | 11450 | 16056 | 8712 | positive |
| 13 | 15770 | 9700 | 4111 | 2335 | 2754 | 1025 | 1001 | 1069 | 558 | positive |
| 14 | 26899 | 20507 | 13152 | 5158 | 34566 | 18887 | 17524 | 24524 | 19103 | positive |
| 15 | 40110 | 22294 | 14237 | 13483 | 23908 | 10768 | 9704 | 12873 | 12875 | positive |
| 16 | 29285 | 14812 | 12227 | 13764 | 5741 | 16013 | 10362 | 7814 | 4958 | positive |
| 17 | 6169 | 2828 | 2472 | 983 | 12809 | 1337 | 543 | 536 | 372 | positive |
| 18 | 10737 | 5860 | 3221 | 847 | 19181 | 3125 | 857 | 912 | 316 | positive |
| 19 | 3372 | 1710 | 1861 | 855 | 14325 | 401 | 228 | 378 | 241 | positive |
| 20 | 4826 | 2600 | 1883 | 859 | 8209 | 459 | 74 | 50 | 48 | positive |
| 21 | 9420 | 4764 | 2473 | 2481 | 18438 | 530 | 420 | 558 | 447 | positive |
| 22 | 3716 | 1157 | 1488 | 687 | 11022 | 3016 | 529 | 245 | 153 | positive |
| 23 | 3962 | 2142 | 1873 | 699 | 16695 | 1965 | 1309 | 559 | 453 | positive |
| 24 | 4829 | 2160 | 2030 | 1818 | 23621 | 8958 | 2658 | 1956 | 1401 | positive |
| 25 | 7940 | 4661 | 2528 | 1071 | 19965 | 6998 | 6377 | 2316 | 1506 | positive |
| 26 | 21004 | 9097 | 5555 | 2889 | 19658 | 3999 | 968 | 655 | 636 | positive |
| 27 | 2887 | 813 | 2055 | 355 | 11946 | 1917 | 256 | 128 | 182 | positive |
| 28 | 25044 | 8379 | 10041 | 14256 | 33037 | 29819 | 23874 | 5817 | 1251 | positive |
| 29 | 18541 | 7548 | 2514 | 544 | 17854 | 14511 | 2512 | 956 | 251 | positive |
| 30 | 13921 | 8514 | 3357 | 1674 | 19126 | 5050 | 1146 | 720 | 446 | positive |
| 31 | 4545 | 1698 | 1672 | 596 | 16584 | 6584 | 3215 | 845 | 605 | positive |
| 32 | 15214 | 7996 | 4562 | 1996 | 21996 | 8965 | 3915 | 1746 | 1748 | positive |
| 33 | 9862 | 4143 | 3326 | 1139 | 13948 | 2438 | 1069 | 586 | 293 | positive |
| 34 | 2985 | 1042 | 1479 | 546 | 4299 | 117 | 69 | 79 | 92 | positive |
| 35 | 1475 | 832 | 641 | 440 | 6033 | 110 | 92 | 93 | 99 | positive |
| 36 | 1023 | 460 | 694 | 312 | 6999 | 1012 | 332 | 123 | 134 | positive |
| 37 | 4667 | 3665 | 2296 | 1050 | 12348 | 627 | 321 | 471 | 109 | positive |
| 38 | 19895 | 7985 | 6985 | 3965 | 31335 | 12691 | 2965 | 2018 | 1824 | positive |
| 39 | 8262 | 3918 | 2217 | 2010 | 12766 | 404 | 152 | 130 | 124 | positive |
| 40 | 8454 | 4512 | 2520 | 905 | 19958 | 1985 | 1081 | 817 | 569 | positive |
| 41 | 4869 | 3154 | 2088 | 1876 | 14431 | 301 | 174 | 108 | 162 | positive |
| 42 | 4418 | 1700 | 2009 | 706 | 17996 | 2020 | 899 | 658 | 570 | positive |
| 43 | 14731 | 8722 | 3526 | 924 | 18234 | 2762 | 1019 | 537 | 489 | positive |
| 44 | 5882 | 2186 | 1850 | 1108 | 10068 | 3715 | 946 | 441 | 257 | positive |
| 45 | 5793 | 3095 | 2070 | 1401 | 23721 | 7457 | 4045 | 1450 | 628 | positive |
| 46 | 1015 | 857 | 755 | 170 | 19449 | 2928 | 2075 | 468 | 826 | positive |
| 47 | 12328 | 7366 | 3145 | 1399 | 22145 | 2921 | 844 | 340 | 215 | positive |
| 48 | 5752 | 2618 | 2431 | 797 | 20449 | 1014 | 661 | 673 | 431 | positive |
| 49 | 8488 | 3424 | 4210 | 1141 | 21470 | 4823 | 4523 | 1853 | 713 | positive |
| 50 | 3989 | 1744 | 1346 | 619 | 18434 | 6315 | 4185 | 1803 | 160 | positive |
| 51 | 903 | 650 | 671 | 291 | 658 | 510 | 110 | 92 | 56 | positive |
| 52 | 6905 | 4325 | 2102 | 1998 | 11120 | 3956 | 2481 | 442 | 517 | positive |
| 53 | 2939 | 2002 | 773 | 440 | 2669 | 542 | 372 | 317 | 385 | positive |
| 54 | 6683 | 3295 | 1408 | 572 | 19008 | 5583 | 3236 | 856 | 813 | positive |
| 55 | 1985 | 549 | 731 | 433 | 716 | 250 | 225 | 234 | 212 | positive |
| 56 | 3228 | 1990 | 940 | 540 | 2070 | 395 | 262 | 166 | 221 | positive |
| 57 | 12985 | 7665 | 2885 | 1655 | 10926 | 3952 | 2365 | 699 | 548 | positive |
| 58 | 6254 | 3452 | 1958 | 1841 | 7985 | 1682 | 676 | 487 | 707 | positive |
| 59 | 6428 | 3858 | 1754 | 1088 | 15985 | 7542 | 4658 | 746 | 699 | positive |
| 60 | 9467 | 8235 | 2547 | 1024 | 17883 | 10764 | 7289 | 1396 | 1127 | positive |
| 61 | 8260 | 6841 | 2403 | 1013 | 11923 | 3895 | 3265 | 372 | 303 | positive |
| 62 | 6341 | 4937 | 2038 | 928 | 6143 | 2699 | 2382 | 720 | 387 | positive |
| 63 | 4812 | 3236 | 1286 | 762 | 2828 | 1311 | 950 | 154 | 188 | positive |
| 64 | 2446 | 1417 | 1388 | 659 | 1542 | 631 | 541 | 138 | 194 | positive |
| 65 | 5896 | 3199 | 1838 | 709 | 1958 | 424 | 197 | 74 | 80 | positive |
| 66 | 13101 | 8985 | 3652 | 1658 | 19856 | 7910 | 5308 | 1921 | 1295 | positive |
| 67 | 5099 | 2003 | 1354 | 784 | 7985 | 2654 | 1995 | 318 | 422 | positive |
| 68 | 8665 | 4980 | 1927 | 1396 | 17168 | 8105 | 5520 | 1026 | 851 | positive |
| 69 | 12542 | 8956 | 499 | 542 | 9584 | 5421 | 2865 | 254 | 310 | positive |
| 70 | 9895 | 4947 | 2695 | 990 | 13114 | 4870 | 2991 | 1819 | 1399 | positive |
| 71 | 7149 | 5658 | 2476 | 2975 | 16003 | 6859 | 3112 | 3722 | 1496 | positive |
| 72 | 6631 | 4666 | 2296 | 1312 | 8465 | 681 | 572 | 310 | 418 | positive |
| 73 | 12220 | 11355 | 4037 | 2916 | 19856 | 4685 | 3378 | 921 | 617 | positive |
| 74 | 9738 | 8170 | 2855 | 1298 | 12710 | 2156 | 1985 | 1254 | 800 | positive |
| 75 | 9965 | 6847 | 2658 | 797 | 14587 | 2654 | 1542 | 554 | 402 | positive |
| 76 | 5948 | 3772 | 1875 | 3695 | 13789 | 1077 | 1069 | 329 | 611 | positive |
| 77 | 7958 | 3889 | 2006 | 744 | 7130 | 386 | 348 | 162 | 202 | positive |
| 78 | 5811 | 4568 | 1935 | 1609 | 6700 | 833 | 659 | 227 | 246 | positive |
| 79 | 3083 | 1738 | 1414 | 1551 | 5630 | 245 | 242 | 169 | 201 | positive |
| 80 | 2546 | 1214 | 1021 | 356 | 1985 | 1021 | 548 | 365 | 212 | positive |
| 81 | 10254 | 8457 | 5468 | 2154 | 15214 | 2452 | 1025 | 566 | 213 | positive |
| 82 | 5621 | 2354 | 1214 | 548 | 14584 | 1744 | 365 | 219 | 145 | positive |
| 83 | 5327 | 4098 | 1977 | 1129 | 6174 | 240 | 390 | 269 | 250 | positive |
| 84 | 2635 | 1966 | 1600 | 691 | 1084 | 167 | 116 | 107 | 77 | positive |
| 85 | 8769 | 6988 | 2371 | 1520 | 9906 | 287 | 235 | 199 | 301 | positive |
| 86 | 1044 | 998 | 788 | 444 | 8955 | 1699 | 842 | 432 | 509 | positive |
| 87 | 6126 | 4416 | 3234 | 2078 | 1326 | 1181 | 86 | 104 | 76 | positive |
| 88 | 6356 | 3455 | 1893 | 1724 | 9098 | 495 | 450 | 141 | 216 | positive |
| 89 | 17917 | 7762 | 4384 | 2328 | 16504 | 3324 | 856 | 576 | 533 | positive |
| 90 | 5435 | 2495 | 2335 | 724 | 18104 | 921 | 607 | 596 | 398 | positive |

NB 20 negative sera were tested; they do not give a fluorescence value greater than the background noise (threshold value=0).

These results show that the use of the test in accordance with the invention allows a sensitive and 100% specific diagnosis of the positive and negative sera, at all the concentrations tested.

As regards the Gp41 antigen, the optimal concentration is 1 mg/ml and 0.5 mg/ml for the Gp120 antigen; however, for the latter, the plateau for saturation of the signal was perhaps not reached, which suggests that higher concentrations could also lead to excellent results.

### EXAMPLE 6

### Study of the Stability Over Time of the Devices Prepared in Examples 4 and 5

In order to test the stability over time of the devices according to the invention, semicarbazide glass slides imprinted by adsorption of AIDS virus antigens, as prepared in example 5 above, were placed at 37° C. in a humid atmosphere for a period of 1 or 3 months. These conditions make it possible to carry out an accelerated aging study.

This study related to slides prepared with various antigens, at several concentrations:

HIV Gp41 at 2; 1; 0.5; 0.1 and 0.05 mg/ml;

HIV Gp120 at 0.5; 0.25; 0.1 and 0.05 mg/ml;

The results obtained are presented in the accompanying FIGS. 1 and 2 in which the fluorescence signal depends on the antigen concentration, these being at 0 (hatched bars), 1 (checkered bars) and 3 months of accelerated aging (plain bars).

These results demonstrate an excellent stability of the slides after 3 months of accelerated aging, regardless of the concentrations tested.

### COMPARATIVE EXAMPLE 7

### Study of the Stability of the Aminated Slides of the Prior Art

Microscope slides silanized with 3-aminopropyl-trimethoxysilane as described for example in the article by Zammatteo N. et al., Anal. Biochem., 2000, 280, 143-150, as well as slides functionalized with semicarbazide groups as described in example 1 above, were imprinted with two AIDS virus antigens (Gp120 and Gp41 as described above in example 5) and at various concentrations (0.5; 0.25; 0.1; 0.05 and 0.01 mg/ml).

The slides thus prepared were incubated with 20 sera noted positive. The slides thus prepared were subjected to a study of stability over time, according to the protocol described above in example 6.

The results obtained are represented in the accompanying FIGS. 3 and 4 in which the fluorescence values are expressed as a function of the concentrations tested.

The stability study was stopped after 30 days (at t=0:clear bars and at t=30 days:shaded bars).

These results show that the aminated slides of the prior art are not stable after 1 month of storage; very large variations are indeed observed with either a drop in the fluorescence measured, or its increase.

On the other hand, and as was demonstrated above in example 6, the slides containing semicarbazide groups in accordance with the invention are stable after three months of storage under accelerated conditions.

### EXAMPLE 8

### Use of Semicarbazide Slides for a Multi Serodetection Study

In this example, semicarbazide glass slides as prepared in example 1 above were imprinted with antigens obtained from various pathologies (HIV, hepatitis B:HBV and hepatitis C:HCV).

90 sera referenced for all these pathologies by conventional ELISA type tests were tested on the slides in accordance with the invention (BIOCHIPS).

For the HIV test, the reference methods used are the Genscreen Plus® HIV Ab test from the company BIORAD and the HIV Integral Ab test from the company BEHRING. The positivity of these tests is confirmed by a Western blotting test.

For the hepatitis B tests, the reference methods used are the HBs test from the company BIORAD and the HBc test from the company BIORAD.

For the hepatitis C tests, the reference methods used are the HCV test from the company BIORAD and the HCV EIA 3.0 test from the company ABBOTT. The positivity is confirmed by a RIBA DECISCAN HCV PLUS® test.

For the hepatitis C test on the biochips in accordance with the invention, the slides were imprinted with the recombinant antigens of NS3 (0.5 mg/ml) and of the whole gene (Batch 1: 0.5 mg/ml).

For the hepatitis B test on the biochips in accordance with the invention, the slides were imprinted with the antigens HBc (core) at 10 mg/ml and HBs Batch b at 5 mg/ml, as described above in example 3.

For the test of seropositivity compared with the AIDS virus on the biochips in accordance with the invention, the slides were imprinted with the Gp120 (0.5 mg/ml) and Gp41 (2 mg/ml) antigens as described above in example 5.

Before their use, the sera were diluted 1/50 as described above. The slides were first incubated for 45 minutes with these sera, and then for 45 minutes with the complementary fluorescent antibody.

The results obtained are presented in table IV below:

TABLE IV

| | | ELISA | | | | BIOCHIP | | | | | |
|---|---|---|---|---|---|---|---|---|---|---|---|
| | Number | | HBV | | | HIV | | HBV | | HCV | |
| Entry | sera | HIV | HBs | HBc | HCV | Gp120 | Gp41 | HBs | HBc | Whole Gene | NS3 |
| 1 | 19 | + | − | − | − | + | + | − | − | − | − |
| 2 | 2 | + | − | + | − | + | + | − | + | − | − |
| 3 | 1 | + | − | − | + | + | + | − | − | + | + |
| 4 | 4 | − | − | − | + | − | − | − | − | − | − |
| 5 | 1 | + | − | − | + | + | + | − | − | − | − |
| 6 | 7 | − | − | + | + | − | − | − | + | + | + |
| 7 | 10 | − | − | − | + | − | − | − | − | + | + |
| 8 | 4 | − | + | + | − | − | − | + | + | − | − |
| 9 | 17 | − | − | + | − | − | − | − | + | − | − |
| 10 | 4 | − | − | + | + | − | − | − | + | + | + |
| 11 | 20 | − | − | − | − | − | − | − | − | − | − |

NB: + positive serum, − negative serum

These results show a perfect correlation for 85 sera tested out of 90; however, for the 5 sera corresponding to the entries 4 and 5, a difference was observed between the two methods, these sera were found to be HCV positive by the reference ELISA method and negative for the BIOCHIP method in accordance with the invention (entries No. 4 and No. 5).

In order to verify this difference, the RIBA test—which serves to confirm the positivity of a serum toward hepatitis C—was carried out and proved to be negative. These sera are therefore HCV negative since they do not possess antibodies directed against the virus, they have not therefore been in contact with this virus.

This confirms the BIOCHIP result which had given these sera as being negative; these sera are therefore false-positives for the reference ELISA method.

### EXAMPLE 9

### Use of Semicarbazide Slides for Assaying the HBs Protein in a Complex Biological Medium

The aim of this example is to demonstrate that the use of the devices in accordance with the invention makes it possible to attach a monoclonal antibody to a glass slide containing semicarbazide groups (antigen directed against the hepatitis B virus surface antigen) which is then brought into contact with a serum possessing this surface antigen; the visualization being carried out using another monoclonal antibody labeled with rhodamine and directed against the hepatitis B virus surface antigen.

This so-called “sandwich” technique makes it possible to detect circulating antigens in a serum. This technique has numerous potential applications both in serodiagnosis and in screening for biological molecules (search for active or toxic substances), by attaching a whole series of known monoclonal antibodies directed against these biological molecules.

1) Materials and Methods

Glass slides functionalized beforehand with semicarbazide groups according to example 1 above are imprinted, using an automated spotter with a solution containing 3 mg/ml of a monoclonal antibody (murine antibody clone NE3 from the company Advanced Immuno Chemical) in a carbonate/bicarbonate buffer (pH 9.2).

Each deposition is performed 3 times, which makes a total of 27 depositions of monoclonal antibody solution for each slide.

Known quantities of a recombinant HBs antigen (company Advanced Immuno Chemical at 0.5 mg/ml) are added to 200 μl of negative sera diluted 1/50. These sera (100 μl) are then brought into contact with the glass slides onto which the monoclonal antibodies have been attached beforehand. The deposits are covered with a glass coverslip. The glass slides are then incubated for 2 hours at 37° C. in a humid atmosphere.

After washing, the visualization step is performed with 100 μl of a solution of murine anti-hepatitis B surface antigen monoclonal antibody (NF5 clone marketed by the company Advanced Immuno Chemical at a concentration of 1.5 mg/ml), labeled beforehand with rhodamine and diluted 1/100 in a dilution solution (PBS-Tween 0.1%—semiskimmed milk 5%).

The incubation is performed for 60 minutes between slide and coverslip, at 37° C. under a humid atmosphere.

After washing, the slides are read by the Scanner from the company Affymetrix (Affymetrix 418 Array) at the power setting P70, PMT90. The quantification of the fluorescence is performed with the aid of the Scanalyse® software (Standford-University Software).

2) Results

The results obtained are presented in the appended FIG. 5 in which the fluorescence is expressed as a function of the quantity of recombinant HBs antigen added in mg/μl.

These results show that the use of the devices in accordance with the invention makes it possible to detect circulating antigens. They also show that the adsorption of the antibody onto the glass slide makes it possible to preserve the accessibility of the site of attachment of the antigen (Fab segment of the antibody) for the antigen-antibody recognition.

1. A device for the presentation of polypeptides, characterized in that it comprises at least one solid support functionalized with semicarbazide groups onto which said polypeptides are adsorbed.

2. The device as claimed in claim 1, characterized in that the polypeptides are chosen from peptides comprising at least two amino acids, peptidomimetics, proteins and protein fragments.

3. The device as claimed in claim 1 or 2, characterized in that the solid support is an organic or inorganic material chosen from glass, silicon and its derivatives and synthetic polymers.

4. A method for preparing a device for the presentation of polypeptides as defined in any one of claims 1 to 4, characterized in that it comprises the following steps:
the functionalization of a solid support with semicarbazide groups, and
the deposition, in the form of spots, of samples of polypeptides and their adsorption onto the support thus functionalized.

5. The method as claimed in claim 4, characterized in that the functionalization of the support comprises:
the introduction of an amine functional group by a reaction for silanization of the support,
the conversion of the amine functional group to an isocyanate functional group,
the reaction of the isocyanate functional group with a hydrazine derivative in order to form the semicarbazide group.

6. The method as claimed in claim 4, characterized in that the functionalization of the support is performed in a single step, by reacting the support with a silane carrying a semicarbazide group.

7. The method as claimed in any one of claims 4 to 6, characterized in that the deposition of the polypeptides comprises:
the preparation of polypeptide solutions in a buffer, at a concentration of between 10 mg/ml and 0.01 mg/ml;
their distribution into a container appropriate for their collection, of the microtiter plate well type;
their collection with the aid of a manual or automated sample collecting apparatus, for example of the “spotter” type;
their deposition onto the semicarbazide support; and optionally
the saturation of the support.

8. The use of a device for the presentation of polypeptides as claimed in any one of claims 1 to 3, as polypeptide chips as a miniaturized and highly parallel diagnostic tool.

9. The use of a device for the presentation of polypeptides as claimed in any one of claims 1 to 3, as polypeptide chips for the detection of a risk during transfusion or organ donation.

10. The use of a device for the presentation of polypeptides as claimed in any one of claims 1 to 3, as polypeptide chips for the serotyping or screening of epitopes.

11. The use as claimed in any one of claims 8 to 10, characterized in that it involves the detection of antigen-antibody type responses by the use of labeled, fluorescent, radioactive or chemically labeled reagents.

12. The use of a device for the presentation of polypeptides as claimed in any one of claims 1 to 3, as polypeptide chips for the quantification of proteins in complex biological media.

13. The use of a device for the presentation of polypeptides as claimed in any one of claims 1 to 3, as polypeptide chips for analyzing the relationships between peptide biological molecules of the ligand-receptor type.
